(19) United States
(12) Patent Application Publication
(10) Pub. No.: US 2023/0045187 A1
(43) Pub. Date: Feb. 9, 2023

(54) COMPOSITIONS COMPRISING A NUCLEASE AND USES THEREOF

(71) Applicant: ARBOR BIOTECHNOLOGIES, Inc., Cambridge, MA (US)

(72) Inventors: David A. Scott, Cambridge, MA (US); Winston X. Yan, Boston, MA (US); David R. Cheng, Boston, MA (US); Tia M. DiTommaso, Waltham, MA (US)

(21) Appl. No.: 17/782,254

(22) PCT Filed: Dec. 3, 2020
(86) PCT No.: PCT/US2020/063125

§ 371 Date: Jun. 3, 2022

Related U.S. Application Data
(60) Provisional application No. 62/943,680, filed on Dec. 4, 2019.

(51) Int. Cl. C12N 9/22; C12N 15/11; C12N 15/90

(52) U.S. Cl. CPC C12N 9/22; C12N 15/11; C12N 15/90; C12N 2310/20; C12N 2800/80

(57) ABSTRACT

The present invention relates to genes coding for nucleases, processes for characterizing the nucleases, cells comprising the nucleases, and methods of using the nucleases.

### CROSS REFERENCE TO RELATED APPLICATION

This application claims the benefit of U.S. Provisional Application No. 62/943,680, filed Dec. 4, 2019. The contents of the aforementioned application is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 2, 2020, is named A2186-7030WO_SL.txt and is 20,769 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although this invention disclosed herein is not limited to specific advantages or functionalities, the invention provides a composition comprising (a) a nuclease or a nucleic acid encoding the nuclease, wherein the nuclease comprises an amino acid sequence with at least 80% identity to SEQ ID NO: 1; and (b) an RNA guide or a nucleic acid encoding the RNA guide, wherein the RNA guide comprises a direct repeat sequence and a spacer sequence, wherein the nuclease binds to the RNA guide, and wherein the spacer sequence binds to a target nucleic acid.

In one aspect of the composition, the nuclease comprises an amino acid sequence set forth in SEQ ID NO: 1.

In another aspect of the composition, the nuclease comprises a RuvC domain or a split RuvC domain.

In another aspect of the composition, the nuclease comprises a catalytic residue (e.g., aspartic acid or glutamic acid).

In another aspect of the composition, the composition does not include a tracrRNA.

In another aspect of the composition, the direct repeat sequence comprises a nucleotide sequence with at least 95% sequence identity to SEQ ID NO: 3 or SEQ ID NO: 4.

In another aspect of the composition, the direct repeat sequence comprises the nucleotide sequence set forth in SEQ ID NO: 3 or SEQ ID NO: 4.

In another aspect of the composition, the spacer sequence comprises between 15 and 24 nucleotides in length.

In another aspect of the composition, the target nucleic acid comprises a sequence complementary to a nucleotide sequence in the spacer sequence.

In another aspect of the composition, the nuclease recognizes a protospacer adjacent motif (PAM) sequence, the PAM sequence comprises a nucleotide sequence set forth as 5′-RTR-3′, 5′-RTG-3′, 5′-NTG-3,′ or 5′-DHD-3′, wherein “R” is A or G, “D” is A or G or T, and “N” is any nucleobase.

In another aspect of the composition, the PAM sequence comprises a nucleotide sequence set forth as 5′-ATG-3′, 5′-GTG-3′, 5′-ATA-3′, or 5′-GTA-3′.

In another aspect of the composition, the nuclease cleaves the target nucleic acid.

In another aspect of the composition, the target nucleic acid is single-stranded DNA or double-stranded DNA.

In another aspect of the composition, the composition comprises at least 10% greater enzymatic activity than a reference composition, e.g., at least 10% greater nuclease activity than a nuclease activity of a reference composition.

In another aspect of the composition, the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In another aspect of the composition, the nucleic acid encoding the nuclease is codon-optimized for expression in a cell.

In another aspect of the composition, the nucleic acid encoding the nuclease is operably linked to a promoter.

In another aspect of the composition, the nucleic acid encoding the nuclease is in a vector.

In another aspect of the composition, the vector comprises a retroviral vector, a lentiviral vector, a phage vector, an adenoviral vector, an adeno-associated vector, or a herpes simplex vector.

In another aspect of the composition, the composition is present in a delivery composition comprising a nanoparticle, a liposome, an exosome, a microvesicle, or a gene-gun.

The invention further provides a cell comprising the composition described herein. In one aspect, the cell is a eukaryotic cell, e.g., a mammalian cell, e.g., a human cell. In another aspect, the cell is a prokaryotic cell.

The invention further provides a method of binding the composition described herein to the target nucleic acid in a cell comprising (a) providing the composition; and (b) delivering the composition to the cell, wherein the cell comprises the target nucleic acid, wherein the nuclease binds to the RNA guide, and wherein the spacer sequence binds to the target nucleic acid.

The invention further provides a method of introducing an insertion or deletion into a target nucleic acid in a cell comprising (a) providing the composition disclosed herein; and (b) delivering the composition to the cell, wherein recognition of the target nucleic acid by the composition results in a modification of the target nucleic acid.

In one aspect of one or more of the methods disclosed herein, delivering the composition to the cell is by transfection.

In another aspect of one or more of the methods, the cell is a eukaryotic cell. In another aspect of one or more of the methods, the cell is a prokaryotic cell. In another aspect of one or more of the methods disclosed herein, the cell is a human cell.

### Definitions

The present invention will be described with respect to particular embodiments and with reference to certain Figures, but the invention is not limited thereto but only by the claims. Terms as set forth hereinafter are generally to be understood in their common sense unless indicated otherwise.

As used herein, the term “catalytic residue” refers to an amino acid that activates catalysis. A catalytic residue is an amino acid that is involved (e.g., directly involved) in catalysis.

As used herein, the terms “domain” and “protein domain” refer to a distinct functional and/or structural unit of a protein. In some embodiments, a domain may comprise a conserved amino acid sequence.

As used herein, the term “enzymatic activity” refers to the catalytic ability of an enzyme. For example, enzymatic activity may include the ability of an enzyme to degrade nucleic acids into shorter oligonucleotides or single nucleotides.

As used herein, the term “nuclease” refers to an enzyme capable of cleaving a phosphodiester bond. A nuclease hydrolyzes phosphodiester bonds in a nucleic acid backbone. As used herein, the term “endonuclease” refers to an enzyme capable of cleaving a phosphodiester bond between nucleotides.

As used herein, the terms “nuclease variant” and “variant nuclease” refer to a nuclease having enzymatic activity and comprising an alteration, e.g., a substitution, insertion, deletion and/or fusion, at one or more (or one or several) positions, compared to its parent sequence.

As used herein, the terms “protospacer adjacent motif” and “PAM sequence” refer to a sequence located near or adjacent to a target sequence. As used herein, a PAM sequence is required for cleavage by a nuclease described herein.

As used herein, the terms “parent,” “nuclease parent,” and “parent sequence” refer to a nuclease to which an alteration is made to produce a variant nuclease of the present invention. In some embodiments, the parent is a nuclease having an identical amino acid sequence of the variant at one or more of specified positions. The parent may be a naturally occurring (wild-type) polypeptide. In a particular embodiment, the parent is a nuclease with at least 60%, at least 61%, at least 62%, at least 63%, at least 64%, at least 65%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% identity to a polypeptide of SEQ ID NO: 1.

As used herein, the terms “reference composition,” “reference sequence,” and “reference” refer to a control, such as a negative control or a parent (e.g., a parent sequence, a parent protein, or a wild-type protein).

As used herein, the terms “RNA guide” or “RNA guide sequence” refer to a molecule that recognizes (e.g., binds to) a target nucleic acid. An RNA guide may be designed to be complementary to a specific nucleic acid sequence. An RNA guide comprises a spacer sequence and a direct repeat (DR) sequence. The terms CRISPR RNA (crRNA), pre-crRNA, mature crRNA, and CRISPR array are also used herein to refer to an RNA guide.

As used herein, the term “RuvC domain” refers to a conserved domain or motif of amino acids having nuclease (e.g., endonuclease) activity. As used herein, a protein having a split RuvC domain refers to a protein having two or more RuvC motifs, at sequentially disparate sites within a sequence, that interact in a tertiary structure to form a RuvC domain.

As used herein, the term “substantially identical” refers to a sequence, polynucleotide, or polypeptide, that has a certain degree of identity to a reference sequence.

As used herein, the terms “target nucleic acid” and “target sequence” refer to a nucleic acid that is specifically bound by a targeting moiety. In some embodiments, the spacer sequence of an RNA guide binds to the target nucleic acid.

As used herein, the terms “trans-activating crRNA” and “tracrRNA” refer to an RNA molecule involved in or required for the binding of an RNA guide to a target nucleic acid.

### BRIEF DESCRIPTION OF THE DRAWINGS

FIG. 1 is a schematic showing the RuvC domain of a canonical Cas12h, with the catalytic residues in the three conserved sequence motifs (I, II, and III) indicated.

FIG. 2A is schematic representation of the components of the negative selection screening assay described in Example 2. CRISPR array libraries were designed to include non-representative spacers uniformly sampled from both strands of the pACYC184 plasmid or E. coli essential genes flanked by two direct repeat sequences and expressed by J23119.

FIG. 2B is a schematic representation of the negative selection screening workflow described in Example 2. CRISPR array libraries were cloned into the effector plasmid (comprising the nuclease described herein). The effector plasmid was transformed into E. coli followed by outgrowth for negative selection of CRISPR arrays conferring interference against transcripts from pACYC184 or E. coli essential genes. Targeted sequencing of the effector plasmid was used to identify depleted CRISPR arrays. Small RNAseq can further be performed to identify mature crRNAs and potential tracrRNA requirements.

FIG. 3A is a graphical representation showing the density of depleted and non-depleted CRISPR arrays for Cas12h1 by location on the pACYC184 plasmid. Targets on the top strand and bottom strand are shown separately and in relation to the orientation of the annotated genes. The magnitude of the bands indicates the degree of depletion, wherein the lighter bands are close to the hit threshold of 3.

FIG. 3B is a graphic representation showing the density of depleted and non-depleted CRISPR arrays for Cas12h1 by location on the DNA of the E. coli strain, E. coli. Targets on the top strand and bottom strand are shown separately and in relation to the orientation of the annotated genes. The magnitude of the bands indicates the degree of depletion, wherein the lighter bands are close to the hit threshold of 3.

FIG. 4 shows sequences flanking depleted targets in E. coli as a prediction of the PAM sequence for Cas12h1.

FIG. 5 shows the predicted secondary structure of a direct repeat sequence of a Cas12h1 guide (SEQ ID NO: 20).

FIG. 6 is a scatter plot that shows the effect of mutating the Cas12h1 RuvC I conserved catalytic residue aspartate (in position 465) to alanine. Each point represents an individual CRISPR array for Cas12h1 or Cas12h1 D465A, and the fold depletion for either CRISPR array was determined from the comparison of the output library to the input library. Higher values indicate stronger depletion (e.g., lack of presence in the output library, e.g., fewer surviving colonies).

FIG. 7A shows a TBE-Urea denaturing gel showing cleavage of dsDNA targets (Target A and Target B) by Cas12h1.

FIG. 7B shows a TBE-Urea denaturing gel showing cleavage of a dsDNA target (Target D) by Cas12h1.

FIG. 7C shows a TBE-Urea denaturing gel showing cleavage of a dsDNA target (Target F) by Cas12h1.

FIG. 8 shows a TBE-Urea denaturing gel showing the following reaction products: target ssDNA (Target G) and Cas12h1, target ssDNA (Target G) and Cas12h1 in complex with a top-strand (active orientation) pre-crRNA, and non-target ssDNA and Cas12h1 in complex with a top-strand (active orientation) pre-crRNA.

FIG. 9A is a schematic showing generation of labeled dsDNA substrates for the dsDNA target cleavage experiments.

FIG. 9B is a schematic showing labeled ssDNA substrates for the ssDNA target cleavage experiments.

### DETAILED DESCRIPTION

The present disclosure relates to a novel nuclease and methods of use thereof. In some aspects, a composition comprising a nuclease having one or more characteristics is described herein. In some aspects, a method of producing the nuclease is described. In some aspects, a method of delivering a composition comprising the nuclease is described.

### Composition

In some aspects, the invention described herein comprises compositions comprising a nuclease. In some embodiments, a composition of the invention includes a nuclease, and the composition has nuclease or endonuclease activity. In some aspects, the invention described herein comprises compositions comprising a nuclease and a targeting moiety. In some embodiments, a composition of the invention includes a nuclease and an RNA guide sequence, and the RNA guide sequence directs the nuclease or endonuclease activity to a site-specific target. In some embodiments, the nuclease is a recombinant nuclease. The nuclease described herein was found in an uncultured metagenomic sequence collected from an aquatic-non marine saline and alkaline-hypersaline lake sediment environment.

In some embodiments, the composition described herein comprises an RNA-guided nuclease (e.g., the nuclease comprises multiple components). In some embodiments, the nuclease comprises enzyme activity (e.g., a protein comprising a RuvC domain or a split RuvC domain). In some embodiments, the composition comprises a targeting moiety (e.g., an RNA guide). In some embodiments, the composition comprises a ribonucleoprotein (RNP) comprising the enzyme moiety and the targeting moiety.

Nuclease

In some embodiments, the composition of the present invention includes a nuclease described herein.

A nucleic acid sequence encoding the nuclease described herein may be substantially identical to a reference nucleic acid sequence if the nucleic acid encoding the nuclease comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the nuclease is encoded by a nucleic acid sequence having at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a reference nucleic acid sequence.

The nuclease described herein may substantially identical to a reference polypeptide if the nuclease comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

In some embodiments, the nuclease of the present invention comprises a polypeptide sequence having 50, 60, 65, 70, 75, 80, 85, 90, 91, 92, 93, 94, 95, 96, 97, 98, 99 or 100% identity to SEQ ID NO: 1. In some embodiments, the nuclease of the present invention comprises a polypeptide sequence having greater than 50, 60, 65, 70, 75, 80, 85, 90, 91, 92, 93, 94, 95, 96, 97, 98, 99 or 100% identity to SEQ ID NO: 1.

In some embodiments, the nuclease of the present invention is a nuclease having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to the amino acid sequence of SEQ ID NO: 1. Homology or identity can be determined by amino acid sequence alignment, e.g., using a program such as BLAST, ALIGN, or CLUSTAL, as described herein.

In some embodiments, the nuclease comprises a protein with an amino acid sequence with at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference amino acid sequence.

Also provided is a nuclease of the present invention having enzymatic activity, e.g., nuclease or endonuclease activity, and comprising an amino acid sequence which differs from the amino acid sequences of any one of SEQ ID NO: 1 by no more than 50, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

In some embodiments, the nuclease comprises a RuvC domain. In some embodiments, the nuclease comprises a split RuvC domain or two or more partial RuvC domains. For example, the nuclease comprises RuvC motifs that are not contiguous with respect to the primary amino acid sequence of the nuclease but form a RuvC domain once the protein folds. In some embodiments, the catalytic residue of a RuvC motif is a glutamic acid residue and/or an aspartic acid residue, including D465 according to the numbering of SEQ ID NO: 1.

In some embodiments, the invention includes an isolated, recombinant, substantially pure, or non-naturally occurring nuclease comprising a RuvC domain, wherein the nuclease has enzymatic activity, e.g., nuclease or endonuclease activity, wherein the nuclease comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 1.

In some embodiments, the invention includes a nuclease comprising a mutated RuvC domain, wherein the nuclease does not have enzymatic activity, e.g., nuclease or endonuclease activity, wherein the nuclease comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to SEQ ID NO: 2.

Biochemical Characteristics

In some embodiments, the biochemistry of the nuclease described herein is analyzed using one or more assays. A pooled screen can be used, as described in Example 2. In this assay, the nuclease of the present invention is cloned and transformed into E. coli along with a CRISPR array library; the CRISPR array library comprises spacers targeting E. coli essential genes or a second plasmid that is co-transformed into E. coli. Analysis of active CRISPR arrays from the pooled screen can be used to determine the activity and PAM sequence preferences of the nucleases described herein. In other embodiments, the biochemistry of the nuclease is analyzed in vitro using a purified nuclease incubated with an RNA guide (e.g., a pre-crRNA) and a target DNA molecule, as described in Examples 7 and 8. The cleavage products are analyzed on a gel.

Described herein are compositions and methods relating to the nuclease. The compositions and methods are based, in part, on the observation that cloned and expressed nucleases of the present invention have nuclease or endonuclease activity.

In some embodiments, a nuclease and an RNA guide as described herein form a complex (e.g., an RNP). In some embodiments, the complex includes other components. In some embodiments, the complex is activated upon binding to a nucleic acid substrate that is complementary to a spacer sequence in the RNA guide (e.g, a target nucleic acid). In some embodiments, the target nucleic acid is a double-stranded DNA (dsDNA). In some embodiments, the target nucleic acid is a single-stranded DNA (ssDNA). In some embodiments, the target nucleic acid is a single-stranded RNA (ssRNA). In some embodiments, the target nucleic acid is a double-stranded RNA (dsRNA). In some embodiments, the sequence-specificity requires a complete match of the spacer sequence in the RNA guide to the target substrate. In other embodiments, the sequence specificity requires a partial (contiguous or non-contiguous) match of the spacer sequence in the RNA guide to the target substrate.

In some embodiments, the complex becomes activated upon binding to the target substrate. In some embodiments, the activated complex exhibits “multiple turnover” activity, whereby upon acting on (e.g., cleaving) the target nucleic acid, the activated complex remains in an activated state. In some embodiments, the activated complex exhibits “single turnover” activity, whereby upon acting on the target nucleic acid, the complex reverts to an inactive state.

In some embodiments, the nuclease described herein binds to a target nucleic acid at a sequence defined by the region of complementarity between the RNA guide and the target nucleic acid. In some embodiments, the PAM sequence of a nuclease described herein is located directly upstream of the target sequence of the target nucleic acid (e.g., directly 5′ of the target sequence). In some embodiments, the PAM sequence of a nuclease described herein is located directly 5′ of the non-complementary strand (e.g., non-target strand) of the target nucleic acid. As used herein, the “complementary strand” hybridizes to the RNA guide. As used herein, the “non-complementary strand” does not directly hybridize to the RNA.

In some embodiments, the PAM sequence of the nuclease described herein is 5′-RTR-3′, 5′-RTG-3′, 5′-NTG-3,′ or 5′-DHD-3′, wherein “R” is A or G, “D” is A or G or T, and “N” is any nucleobase. In some embodiments, the PAM sequence comprises a nucleotide sequence set forth as 5′-ATG-3′, 5′-GTG-3′, 5′-ATA-3′, or 5′-GTA-3′.

In some embodiments, the nuclease described herein cleaves ssDNA. In some embodiments, the nuclease described herein cleaves dsDNA. In some embodiments, the nuclease described herein is a nickase (e.g., the nuclease cleaves one strand of a double-stranded target nucleic acid).

In some embodiments, the nuclease of the present invention has enzymatic activity, e.g., nuclease or endonuclease activity, over a broad range of pH conditions. In some embodiments, the nuclease has enzymatic activity, e.g., nuclease or endonuclease activity, at a pH of from about 3.0 to about 12.0. In some embodiments, the nuclease has enzymatic activity at a pH of from about 4.0 to about 10.5. In some embodiments, the nuclease has enzymatic activity at a pH of from about 5.5 to about 8.5. In some embodiments, the nuclease has enzymatic activity at a pH of from about 6.0 to about 8.0. In some embodiments, the nuclease has enzymatic activity at a pH of about 7.0.

In some embodiments, the nuclease of the present invention has enzymatic activity, e.g., nuclease or endonuclease activity, at a temperature range of from about 10° C. to about 100° C. In some embodiments, the nuclease of the present invention has enzymatic activity at a temperature range from about 20° C. to about 90° C. In some embodiments, the nuclease of the present invention has enzymatic activity at a temperature of about 20° C. to about 25° C. or at a temperature of about 37° C.

Variants

In some embodiments, the present invention includes variants of the nuclease described herein. In some embodiments, the nuclease described herein can be mutated at one or more amino acid residues to modify one or more functional activities. For example, in some embodiments, the nuclease is mutated at one or more amino acid residues to modify its nuclease activity (e.g., cleavage activity). For example, in some embodiments, the nuclease may comprise one or more mutations that increase the ability of the nuclease to cleave a target nucleic acid. In some embodiments, the nuclease is mutated at one or more amino acid residues to modify its ability to functionally associate with an RNA guide. In some embodiments, the nuclease is mutated at one or more amino acid residues to modify its ability to functionally associate with a target nucleic acid. In some embodiments, the nuclease further has helicase activity and is mutated at one or more amino acid residues to modify its helicase activity.

In some embodiments, a variant nuclease has a conservative or non-conservative amino acid substitution, deletion or addition. In some embodiments, the variant nuclease has a silent substitution, deletion or addition, or a conservative substitution, none of which alter the polypeptide activity of the present invention. Typical examples of the conservative substitution include substitution whereby one amino acid is exchanged for another, such as exchange among aliphatic amino acids Ala, Val, Leu and Ile, exchange between hydroxyl residues Ser and Thr, exchange between acidic residues Asp and Glu, substitution between amide residues Asn and Gln, exchange between basic residues Lys and Arg, and substitution between aromatic residues Phe and Tyr. In some embodiments, one or more residues of a nuclease disclosed herein are mutated to an Arg residue. In some embodiments, one or more residues of a nuclease disclosed herein are mutated to a Gly residue.

A variety of methods are known in the art that are suitable for generating modified polynucleotides that encode variant nucleases of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., nucleases) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60) [2001]), and NRR (See, Bittker et al., 20:1024-9 [2001]; Bittker et al., 101:7011-6 [2004]), and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

In some embodiments, the nuclease comprises an alteration at one or more (e.g., several) amino acids in the nuclease, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more.

As used herein, a “biologically active portion” is a portion that maintains the function (e.g. completely, partially, minimally) of the nuclease (e.g., a “minimal” or “core” domain). In some embodiments, a nuclease fusion protein is useful in the methods described herein. Accordingly, in some embodiments, a nucleic acid encoding the fusion nuclease is described herein. In some embodiments, all or a portion of one or more components of the nuclease fusion protein are encoded in a single nucleic acid sequence.

Although the changes described herein may be one or more amino acid changes, changes to the nuclease may also be of a substantive nature, such as fusion of polypeptides as amino- and/or carboxyl-terminal extensions. For example, nuclease may contain additional peptides, e.g., one or more peptides. Examples of additional peptides may include epitope peptides for labelling, such as a polyhistidine tag (His-tag), Myc, and FLAG. In some embodiments, a nuclease described herein can be fused to a detectable moiety such as a fluorescent protein (e.g., green fluorescent protein (GFP) or yellow fluorescent protein (YFP)).

The nuclease described herein can be modified to have diminished nuclease activity, e.g., nuclease inactivation of at least 50%, at least 60%, at least 70%, at least 80%, at least 90%, at least 95%, at least 97%, or 100%, as compared to a reference nuclease. The nuclease activity can be diminished by several methods known in the art, e.g., introducing mutations into the RuvC domain (e.g, one or more catalytic residues of the RuvC domain). A non-limiting example of an inactivated nuclease (e.g., a RuvC mutant) is set forth in SEQ ID NO: 2.

In some embodiments, the nuclease described herein can be self-inactivating. See, Epstein et al., “Engineering a Self-Inactivating CRISPR System for AAV Vectors,” Mol. Ther., 24 (2016): S50, which is incorporated by reference in its entirety.

Nucleic acid molecules encoding the nucleases described herein can further be codon-optimized. The nucleic acid can be codon-optimized for use in a particular host cell.

Targeting Moiety

In some embodiments, the composition described herein comprises a targeting moiety.

The targeting moiety may be substantially identical to a reference nucleic acid sequence if the targeting moiety comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the targeting moiety has at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence.

RNA Guide Sequence

In some embodiments, the targeting moiety comprises, or is, an RNA guide sequence. In some embodiments, the RNA guide sequence directs the nuclease described herein to a particular nucleic acid sequence. Those skilled in the art reading the below examples of particular kinds of RNA guide sequences will understand that, in some embodiments, an RNA guide sequence is site-specific. That is, in some embodiments, an RNA guide sequence associates specifically with one or more target nucleic acid sequences (e.g., specific DNA or genomic DNA sequences) and not to non-targeted nucleic acid sequences (e.g., non-specific DNA or random sequences).

In some embodiments, the composition as described herein comprises an RNA guide sequence that associates with nuclease described herein and directs the nuclease to a target nucleic acid sequence (e.g., DNA). The RNA guide sequence may associate with a nucleic acid sequence and alter functionality of the nuclease (e.g., alters affinity of the nuclease to a molecule, e.g., at least 10%, 15%, 20%, 25%, 30%, 35%, 40%, 45%, 50%, 55%, 60%, 65%, 70%, 75%, 80%, 85%, 90%, 95%, or more).

The RNA guide sequence may target (e.g., associate with, be directed to, contact, or bind) one or more nucleotides of a sequence, e.g., a site-specific sequence or a site-specific target. In some embodiments, the nuclease (e.g., a nuclease plus an RNA guide) is activated upon binding to a nucleic acid substrate that is complementary to a spacer sequence in the RNA guide (e.g., a sequence-specific substrate or target nucleic acid).

In some embodiments, an RNA guide sequence comprises a spacer sequence. In some embodiments, the spacer sequence of the RNA guide sequence may be generally designed to have a length of between 17-24 nucleotides (e.g., 19, 20, or 21 nucleotides) and be complementary to a specific nucleic acid sequence. In some particular embodiments, the RNA guide sequence may be designed to be complementary to a specific DNA strand, e.g., of a genomic locus. In some embodiments, the spacer sequence is designed to be complementary to a specific DNA strand, e.g., of a genomic locus.

In certain embodiments, the RNA guide sequence includes, consists essentially of, or comprises a direct repeat sequence linked to a sequence or spacer sequence. In some embodiments, the RNA guide sequence includes a direct repeat sequence and a spacer sequence or a direct repeat-spacer-direct repeat sequence. In some embodiments, the RNA guide sequence includes a truncated direct repeat sequence and a spacer sequence, which is typical of processed or mature crRNA. In some embodiments, the nuclease forms a complex with the RNA guide sequence, and the RNA guide sequence directs the complex to associate with site-specific target nucleic acid that is complementary to at least a portion of the RNA guide sequence. In some embodiments, the RNA guide sequence does not include a tracrRNA.

In some embodiments, the RNA guide sequence comprises a sequence, e.g., RNA sequence, at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a DNA sequence. In some embodiments, the RNA guide sequence comprises a sequence at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a genomic sequence. In some embodiments, the RNA guide sequence comprises a sequence complementary to or a sequence comprising at least 80%, at least 90%, at least 95%, at least 96%, at least 97%, at least 98%, at least 99% complementarity to a genomic sequence.

In some embodiments, the nuclease described herein includes one or more (e.g., two, three, four, five, six, seven, eight, or more) RNA guide sequences, e.g., RNA guides.

In some embodiments, the RNA guide has an architecture similar to, for example International Publication Nos. WO 2014/093622 and WO 2015/070083, the entire contents of each of which are incorporated herein by reference.

In some embodiments, an RNA guide sequence of the present invention comprises a direct repeat sequence having 80, 85, 90, 91, 92, 93, 94, 95, 96, 97, 98, 99 or 100% identity to SEQ ID NO: 3 or SEQ ID NO: 4. In some embodiments, the targeting moiety of the present invention comprises a direct repeat sequence having greater than 80, 85, 90, 91, 92, 93, 94, 95, 96, 97, 98, 99 or 100% identity to SEQ ID NO: 3 or SEQ ID NO: 4.

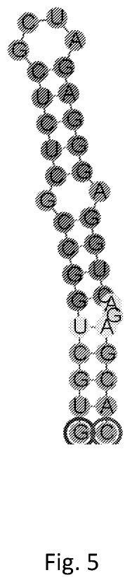

In some embodiments, a direct repeat of an RNA guide sequence of the present invention comprises a stem-loop structure, as shown in FIG. 5. In some embodiments, a direct repeat sequence having 80, 85, 90, 91, 92, 93, 94, 95, 96, 97, 98, 99 or 100% identity to SEQ ID NO: 3 or SEQ ID NO: 4 comprises a stem-loop structure.

Non-limiting examples of pre-crRNA sequences capable of being utilized by the nuclease described herein can be found in SEQ ID NOs: 6, 9, 12, 15, and 18. In some embodiments, a nuclease described herein in combination with a pre-crRNA of any one of SEQ ID NO: 6, SEQ ID NO: 9, SEQ ID NO: 12, SEQ ID NO: 15, and SEQ ID NO: 18 has nuclease activity (e.g., cleaves a site-specific target nucleic acid set forth in SEQ ID NO: 5, SEQ ID NO: 8, SEQ ID NO: 11, SEQ ID NO: 14, and SEQ ID NO: 17, respectively). In some embodiments, a nuclease in combination with a pre-crRNA having at least 80, 85, 90, 91, 92, 93, 94, 95, 96, 97, 98, 99 or 100% identity of any one of SEQ ID NO: 6, SEQ ID NO: 9, SEQ ID NO: 12, SEQ ID NO: 15, and SEQ ID NO: 18 has nuclease activity (e.g., cleaves a site-specific target nucleic acid).

Unless otherwise noted, all compositions and nucleases provided herein are made in reference to the active level of that composition or nuclease, and are exclusive of impurities, for example, residual solvents or by-products, which may be present in commercially available sources. Nuclease component weights are based on total active protein. All percentages and ratios are calculated by weight unless otherwise indicated. All percentages and ratios are calculated based on the total composition unless otherwise indicated. In the exemplified composition, the nuclease levels are expressed by pure enzyme by weight of the total composition and unless otherwise specified, the ingredients are expressed by weight of the total compositions.

### Modifications

The RNA guide sequence or any of the nucleic acid sequences encoding the nuclease may include one or more covalent modifications with respect to a reference sequence, in particular the parent polyribonucleotide, which are included within the scope of this invention. Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide sequence or any of the nucleic acid sequences encoding components of the nuclease may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Vectors

The present invention provides a vector for expressing the nuclease described herein or nucleic acids encoding the nuclease described herein may be incorporated into a vector. In some embodiments, a vector of the invention includes a nucleotide sequence encoding the nuclease, e.g., one or more components of the nuclease. In some embodiments, a vector of the invention includes a nucleotide sequence encoding the nuclease.

The present invention also provides a vector that may be used for preparation of the nuclease or compositions comprising the nuclease as described herein. In some embodiments, the invention includes the composition or vector described herein in a cell. In some embodiments, the invention includes a method of expressing the composition comprising the nuclease, or vector or nucleic acid encoding the nuclease, in a cell. The method may comprise the steps of providing the composition, e.g., vector or nucleic acid, and delivering the composition to the cell. Expression of natural or synthetic polynucleotides is typically achieved by operably linking a polynucleotide encoding the gene of interest to a promoter and incorporating the construct into an expression vector. The expression vector is not particularly limited as long as it includes a polynucleotide encoding the nuclease of the present invention and can be suitable for replication and integration in eukaryotic cells. Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the nuclease from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the nuclease of the present invention has been transferred into the host cells and then expressed without fail.

The preparation method for recombinant expression vectors is not particularly limited, and examples thereof include methods using a plasmid, a phage or a cosmid.

### Production

In some embodiments, the nuclease of the present invention can be prepared by (I) culturing bacteria which produce the nuclease of the present invention, isolating the nuclease, and optionally, purifying the nuclease. The nuclease can be also prepared by (II) a known genetic engineering technique, specifically, by isolating a gene encoding the nuclease of the present invention from bacteria, constructing a recombinant expression vector, and then transferring the vector into an appropriate host cell for expression of a recombinant protein. Alternatively, the nuclease can be prepared by (III) an in vitro coupled transcription-translation system. Bacteria that can be used for preparation of the nuclease of the present invention are not particularly limited as long as they can produce the nuclease of the present invention. Some nonlimiting examples of the bacteria include E. coli cells described herein.

Methods of Expression

The present invention includes a method for protein expression, comprising translating the nuclease described herein.

In some embodiments, a host cell described herein is used to express the nuclease. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of the nuclease. After expression of the nuclease, the host cells can be collected and nuclease purified from the cultures etc. according to conventional methods (for example, filtration, centrifugation, cell disruption, gel filtration chromatography, ion exchange chromatography, etc.).

In some embodiments, the methods for nuclease expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of the nuclease. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of the nuclease.

A variety of methods can be used to determine the level of production of a mature nuclease in a host cell. Such methods include, but are not limited to, for example, methods that utilize either polyclonal or monoclonal antibodies specific for the nuclease. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of the nuclease in a cell, comprising providing a polyribonucleotide encoding the nuclease to a host cell wherein the polyribonucleotide encodes the nuclease, expressing the nuclease in the cell, and obtaining the nuclease from the cell.

### Delivery

Compositions described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Sequence of Cas12h1 Nuclease

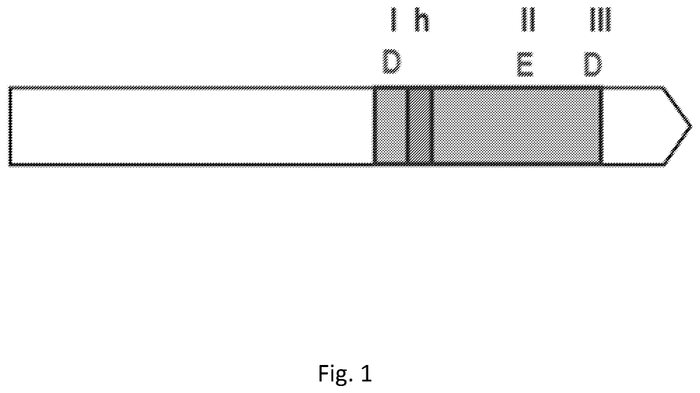

In this Example, amino acid sequences of Cas12h family members were analyzed to identify potential functional protein domains. As shown in FIG. 1, the amino acid sequences were determined to include a putative C-terminal RuvC domain. The catalytic residues were also determined to reside in conserved sequence motifs (I, II, and III) of the RuvC domain. The sequence was further determined to include a bridge helix (h) domain.

This Example indicates that the amino acid sequences of the Cas12h family members were shown to have a conserved C-terminal domain RuvC domain.

### Example 2—In vivo Analysis of Engineered Cas12h1 System

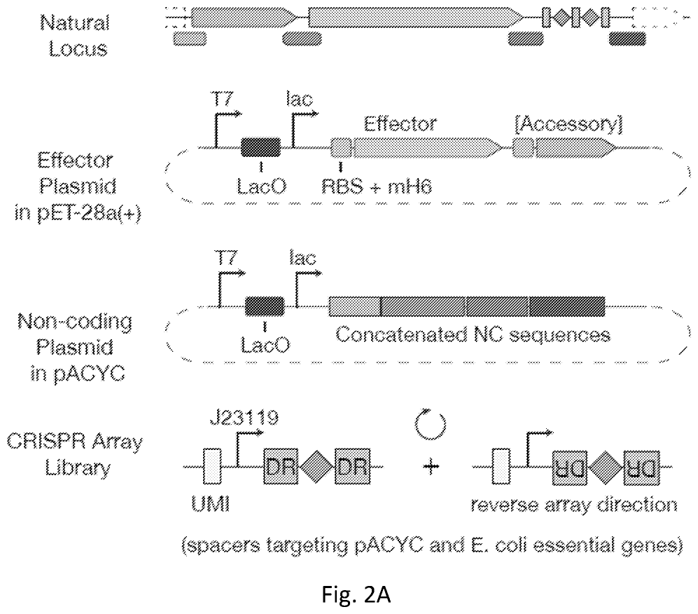

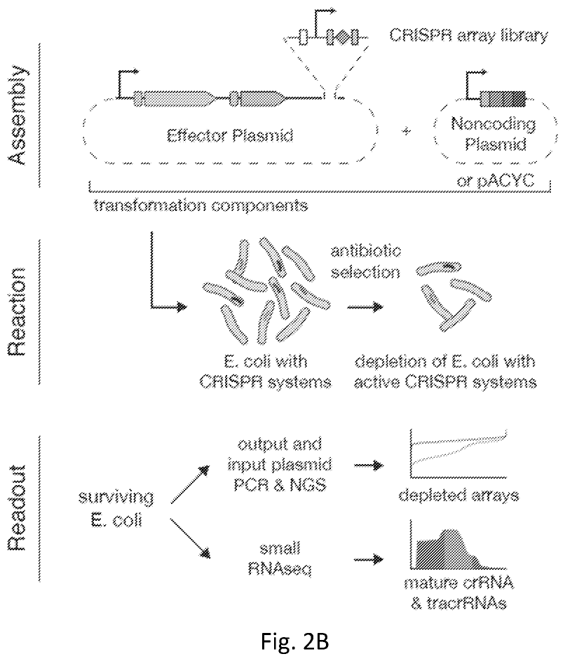

In this Example, a Cas12h1 system was engineered and tested in an E. coli system. The Cas12h1 nuclease (SEQ ID NO: 1) was E. coli codon-optimized, synthesized (Genscript) and cloned into a custom expression system derived from pET-28a(+) (EMD-Millipore). The vector included a nucleic acid encoding Cas12h1 under the control of a lac promoter and an E. coli ribosome binding sequence. The vector also included an acceptor site for a CRISPR array library driven by a J23119 promoter following the open reading frame for Cas12h1. See FIG. 2A.

An oligonucleotide library synthesis (OLS) pool containing direct repeat-spacer-direct repeat sequences was computationally designed, where the direct repeat represents a consensus direct repeat sequence found in the CRISPR array associated with the natural Cas12h1 locus, and the spacer represents a sequence tiling the pACYC184 plasmid comprising chloramphenicol and tetracycline resistance genes, E. coli essential genes, or a negative control sequence (GFP). In particular, the direct repeat sequence in each library for Cas12h1 was the sequence of SEQ ID NO: 3 or SEQ ID NO: 4. The spacer length was determined by the mode of the spacer lengths found in the endogenous CRISPR array. Redundant direct repeat sequences were represented in the library that tile the pACYC184 plasmid, E. coli essential genes, or negative control sequence to provide internal controls. An individual direct repeat-spacer-direct repeat sequence is also described as a CRISPR array in these Examples.

The library of targeting CRISPR array sequences was next cloned into the Cas12h1 plasmid to create a Cas12h1/CRISPR array library. Flanking restriction sites, a unique molecular identifier (barcode), unique PCR priming sites for specific amplification of the targeting library from the larger pool, and a J23119 promoter were appended to the targeting library using PCR (NEBNext High-Fidelity 2×PCR Master Mix), and then an optimized restriction enzyme and ligase (New England Biolabs) was added to generate the Cas12h1/CRISPR array library. This represented the input library for the screen.

Next, E. coli were transformed with the Cas12h1/CRISPR array library. The cells were electroporated with the input library according to the manufacturer's protocols using an electroporation system (Bio-rad) with a 1.0 mm cuvette. The cells were plated onto bioassay plates with both chloramphenicol (Fisher) and kanamycin (Alfa Aesar) and grown for 11 hours. Subsequently, the approximate colony count was estimated to ensure sufficient library representation, and the cells were harvested. See FIG. 2B.

Cells transformed with Cas12h1/CRISPR array library were grown, harvested, and analyzed. Plasmid DNA fractions were extracted from the harvested cells to create the output library using a DNA prep kit (Qiagen), while total RNA was harvested by processing the harvested cells with an RNA purification kit (Zymo Research), followed by extraction using an RNA prep kit (Zymo Research).

A proxy for activity of the engineered Cas12h1/CRISPR array library in E. coli was investigated, wherein bacterial cell death was used as the proxy for Cas12h1 activity. An active Cas12h1 enzyme associated with a CRISPR array sequence could selectively bind and disrupt expression of a spacer sequence target, e.g., pACYC184 plasmid or E. coli essential gene, resulting in cell death, thereby depleting representation of this specific CRISPR array in the output library, as opposed to the input library.

A next generation sequencing (NGS) library for detecting those CRISPR arrays depleted from the output library, as compared to the input library, was prepared by performing PCR on both the input and output libraries, using the unique primers that flank the targeting library of the CRISPR array to identify each CRISPR array sequence by the barcodes. The library was then normalized, pooled, and loaded onto a high-throughput sequence system (Illumina) to evaluate the presence (and absence) of barcodes.

NGS data for screening input and output libraries were demultiplexed using software to convert base call files into FASTQ files. Reads for each sample included information about the targeting library in the screening. The direct repeat sequence of each targeting CRISPR array sequence was used to determine the direct repeat-spacer-direct repeat sequence orientation, and the spacer sequence was mapped to the source (pACYC184 or E. coli essential genes) or negative control sequence (GFP) to determine the corresponding target. For each sample, the total number of reads for each CRISPR array sequence (rₐ) in a given output library was counted and normalized as follows: (rₐ+1)/total reads for all CRISPR array library elements. The depletion score was calculated by dividing normalized output reads for a given CRISPR array by normalized input reads.

Fold depletion for each CRISPR array was defined as the normalized input read count divided by the normalized output read count (with 1 added to avoid division by zero). A CRISPR array was considered to be strongly depleted if the fold depletion was greater than 3. When calculating the CRISPR array fold-depletion for Cas12h1 across biological replicates, the maximum fold depletion value for a given CRISPR array across all experiments (i.e., a strongly depleted CRISPR array must be strongly depleted in all biological replicates) was taken.

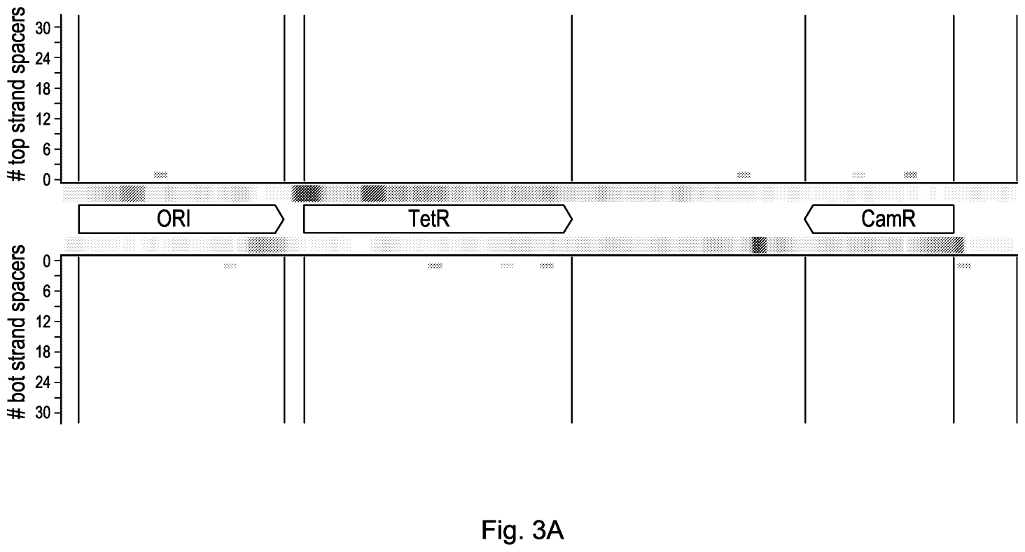

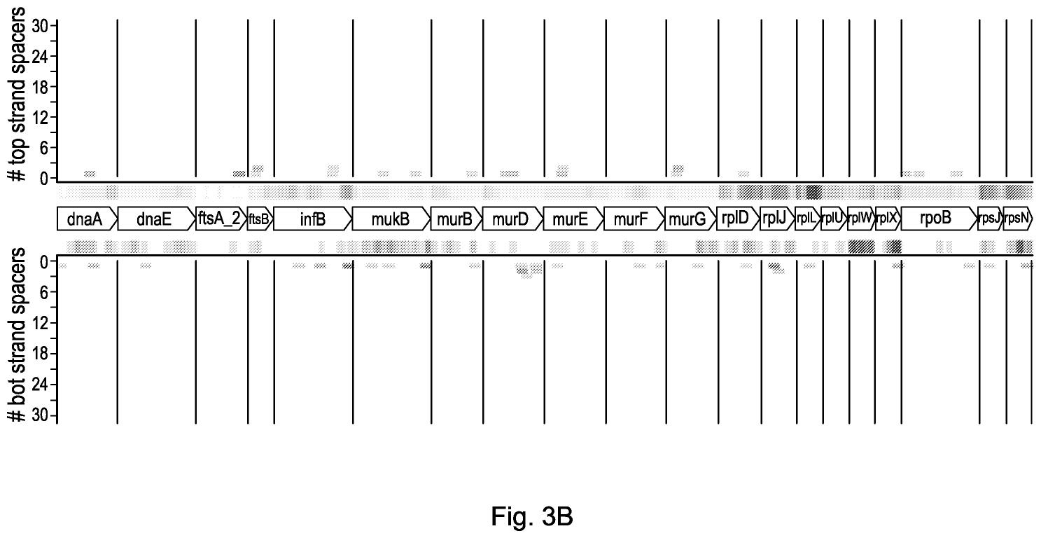

FIG. 3A and FIG. 3B depict the locations in the pACYC184 plasmid and E. coli essential genes, respectively, that the CRISPR arrays targeted. The locations of the plasmid or gene targets were found to be dispersed throughout with little preference for the top or bottom strands.

This Example indicates that the CRISPR arrays associated with Cas12h1 targeted and disrupted expression in E. coli.

### Example 3—Identification of PAM Sequence for Cas12h1

In this Example, identification of PAM sequences was performed.

The depleted CRISPR array sequences depicted in FIG. 3A and FIG. 3B were aligned to identify potential sequence requirements for Cas12h1 CRISPR systems.

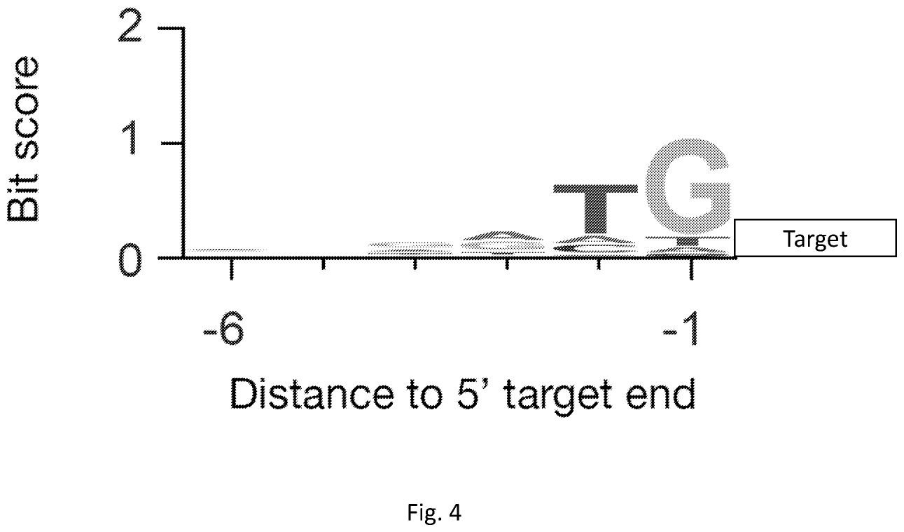

FIG. 4 shows a preference of PAM sequences flanking the target spacer sequences in E. coli. This analysis revealed possible PAM sequences of 5′-TG-3′, 5′-RTG-3′, and 5′-RTR-3′ for Cas12h1.

This Example suggests that Cas12h1 interaction with target DNA may be PAM-dependent.

### Example 4—Predicted Secondary Structure of Direct Repeat Sequence of Cas12h1 RNA Guide

This Example describes a predicted secondary structure for a Cas12h1 RNA guide sequence.

In this Example, the sequence of a direct repeat sequence of a Cas12h1 RNA guide (SEQ ID NO: 3) was analyzed for its predicted secondary structure. As shown in FIG. 5, the predicted folding of the direct repeat sequence suggested a stem-loop structure. The RNA free energy was calculated to be −18.7 kcal/mol.

This Example suggests that the stem-loop structure of the Cas12h1 RNA guide direct repeat sequence was energetically favored.

### Example 5—Cas12h1 RuvC Mutant System in E. coli

In this Example, a Cas12h1 RuvC mutant was designed and tested in an E. coli system.

A conserved catalytic residue in the Cas12h1 RuvC I motif domain (in location 465) was mutated to alanine by site-directed mutagenesis (D465A). The Cas12h1 D465A sequence is set forth in SEQ ID NO: 2. The vector included the nucleic acid encoding Cas12h1 D465A under the control of a lac promoter and an E. coli ribosome binding sequence. The vector also included an acceptor site for a targeting library driven by a J23119 promoter following the open reading frame for Cas12h1 D465A. The CRISPR array library (direct repeat-spacer-direct repeat library) was next cloned into the Cas12h1 D465A plasmid, and the Cas12h1 D465A/CRISPR array library was transformed into E. coli as described in Example 2.

Cells were grown, harvested and analyzed by NGS as described in Example 2.

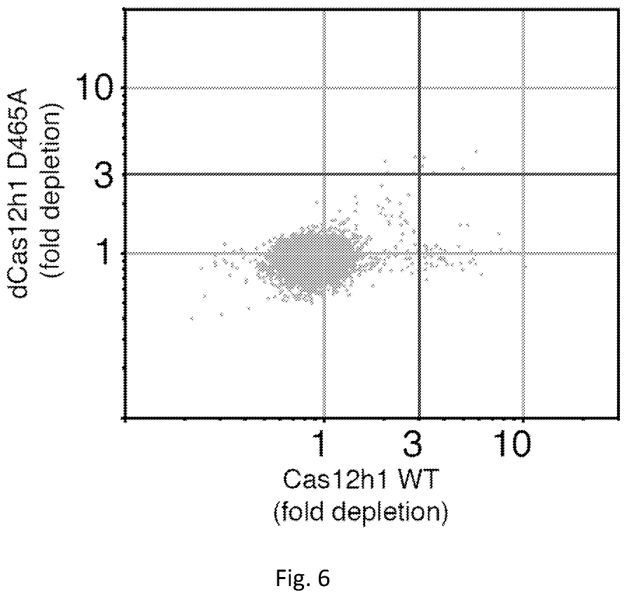

FIG. 6 is a scatter plot, wherein each point represents an individual CRISPR array associated with Cas12h1 or Cas12h1 D465A, and the fold-depletion for either the wild-type or the mutant Cas12h1 was determined from the comparison of the output library to the input library. Higher values indicate stronger depletion (e.g., lack of presence in the output library, e.g., fewer surviving colonies). As shown in FIG. 6, wild-type Cas12h1 (SEQ ID NO: 1) demonstrated higher numbers of CRISPR arrays depleted in the output library, as compared to the depletion with the Cas12h1 D465A mutant (SEQ ID NO: 2).

This Example suggests that the Cas12h1 mutant demonstrated less depletion of CRISPR arrays than the wild-type Cas12h1.

### Example 6—Purification of Cas12h1 Protein

In this Example, Cas12h1 was purified for biochemical testing of Cas12h1.

The plasmid comprising Cas12h1 from Example 2 was transformed into E. coli cells (New England BioLabs) and expressed under a T7 promoter. Transformed cells were initially grown overnight in 3 mL Luria Broth (Sigma)+50 μg/mL kanamycin, followed by inoculation of 1 L of media (Sigma)+50 μg/mL kanamycin with 1 mL of overnight culture. Cells were grown at 37° C. to an OD₆₀₀ of 1-1.5, then protein expression was induced with 0.2 mM IPTG. Cultures were then grown at 20° C. for an additional 14-18 h.

Cultures were harvested and pelleted via centrifugation, then resuspended in 80 mL of lysis buffer (50 mM HEPES pH 7.6, 0.5 M NaCl, 10 mM imidazole, 14 mM 2-mercaptoethanol, and 5% glycerol)+protease inhibitors (Sigma). Cells were lysed via cell disruptor (Constant System Limited), then centrifuged twice at 28,000×g for 20 min at 4° C. in order to clarify the lysate.

The lysate was loaded onto a 5 mL HisTrap FF column (GE Life Sciences), then purified via FPLC (AKTA Pure, GE Life Sciences) over an imidazole gradient from 10 mM to 250 mM. Cas12h1 was eluted in low salt buffer (50 mM HEPES-KOH pH 7.8, 500 mM NaCl, 10 mM MgCl₂, 14 mM mercaptoethanol, and 5% glycerol). After elution, fractions were run on SDS-PAGE gels, and fractions containing protein of the appropriate size were pooled and concentrated using 10 kD Amicon Ultra-15 Centrifugal Units. Cas12h1 was further dialyzed into a buffer without imidazole (25 mM HEPES-KOH pH 7.8, 500 mM NaCl, 10 mM MgCl2, 1 mM DTT, 7 mM 2-mercaptoethanol, and 30% glycerol). Protein concentration was determined by Qubit protein assay (Thermo Fisher).

### Example 7—dsDNA Cleavage with Cas12h1

This Example demonstrates biochemical testing of Cas12h1.

Using information obtained from Example 2, RNA guide sequences were synthesized for Cas12h1. Spacer sequences of the pre-crRNA were generated for complementarity to one strand of a DNA target for cleavage testing.

The pre-crRNA (or RNA guide) sequences for Cas12h1 were prepared using in vitro transcription (IVT). T7 promoter containing double-stranded DNA templates for pre-crRNAs were prepared using PCR (NEBNEXT High-fidelity 2×PCR Master Mix, NEB). IVT was performed by incubating the double-stranded DNA templates with T7 RNA polymerase (HiScribe T7 Quick Hihg Yield RNA synthesis kit NEB) followed by treatment with DNase (Thermo Fisher Scientific) to remove the DNA template. The IVT product was cleaned up using RNA prep kit (Zymo Research).

Table 1 shows sequence identifiers for targets A, B, D, F, and G and their corresponding pre-crRNA (direct repeat-spacer-direct repeat) and spacer sequences. Targets A, B, D, F, and G correspond to different sequences within GFP.

TABLE 1
SEQ ID NOs for assays described below

| | Target | pre-crRNA | Spacer |
|---|---|---|---|
| Target | Sequence | Sequence | Sequence |
| A | SEQ ID NO: 5 | SEQ ID NO: 6 | SEQ ID NO: 7 |
| B | SEQ ID NO: 8 | SEQ ID NO: 9 | SEQ ID NO: 10 |
| D | SEQ ID NO: 11 | SEQ ID NO: 12 | SEQ ID NO: 13 |
| F | SEQ ID NO: 14 | SEQ ID NO: 15 | SEQ ID NO: 16 |
| G | SEQ ID NO: 17 | SEQ ID NO: 18 | SEQ ID NO: 19 |

ssDNA and dsDNA target sequences were synthesized for Cas12h1 biochemical testing. One strand of the dsDNA target was complementary to the spacer sequence described above.

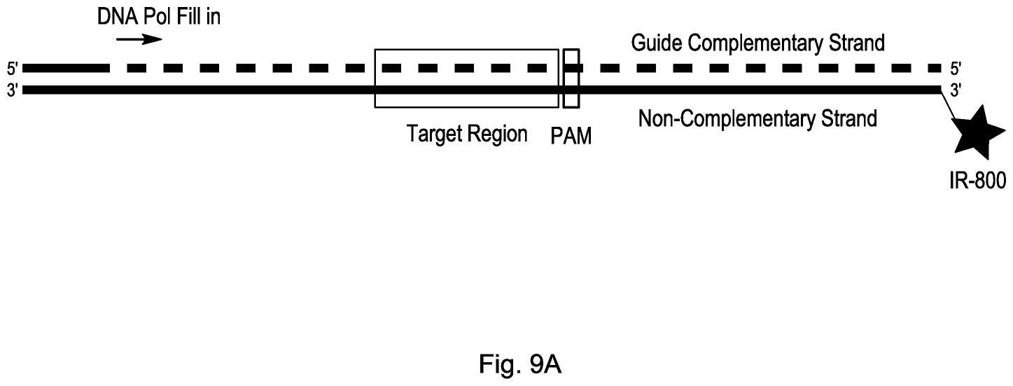

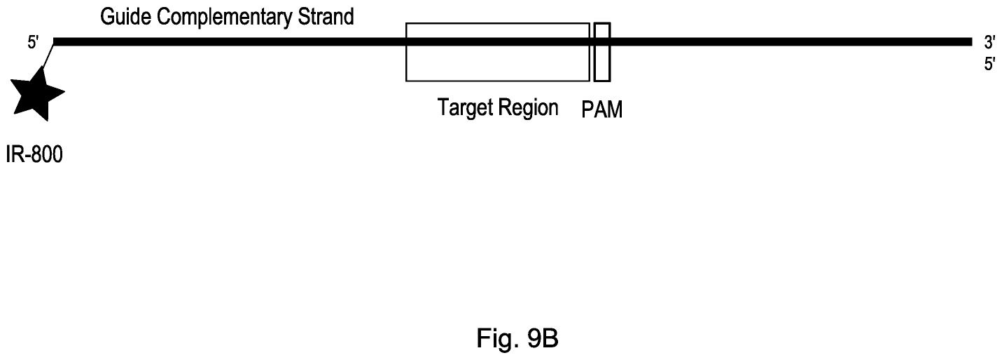

Labeled dsDNA target substrates were generated by labeling the non-spacer complementary (NSC) strand, annealing with a primer, then extending with DNA Polymerase I (New England BioLabs), as shown in FIG. 9A. These substrates were purified with DNA prep kit (Zymo Research). Concentrations were measured (Thermo Fisher Scientific). The NSC strands of the dsDNA targets were labelled with near-infrared fluorescent dye using 5′ labeling kit (Vector Labs) and following the manufacturer's protocol. ssDNA oligos containing the target complementary region were synthesized commercially (IDT) and labelled with near-infrared fluorescent dye using 5′ labeling kit (Vector Labs) following the manufacturer's protocol.

Cas12h1 was tested for specific activity across 4 different targets: Target A, B, D, and F. Negative controls with no Cas12h1 and non-targeting pre-crRNAs (e.g., using RNA guide designed for Target A with Target B, etc.) were also tested. dsDNA target cleavage assays were set up in a reaction buffer (50 mM NaCl, 10 mM Tris, 10 mM MgCl₂, 1 mM DTT, pH 8.0). Complexed RNPs (Cas12h1 with pre-crRNAs) were formed by incubating purified Cas12h1 from Example 6 with the pre-crRNAs from Table 1 or non-targeting pre-crRNAs at a ratio of 1:2. Complexed RNPs were then added to 100 nM dsDNA substrate and incubated. Reactions were treated with an RNase cocktail and incubated. Next, the reactions were treated with Proteinase K and incubated.

To detect dsDNA cleavage, DNA products from the reactions were analyzed on 15% TBE-Urea gels. Gels were imaged on a fluorescent digital imaging system (LI-COR Biosciences).

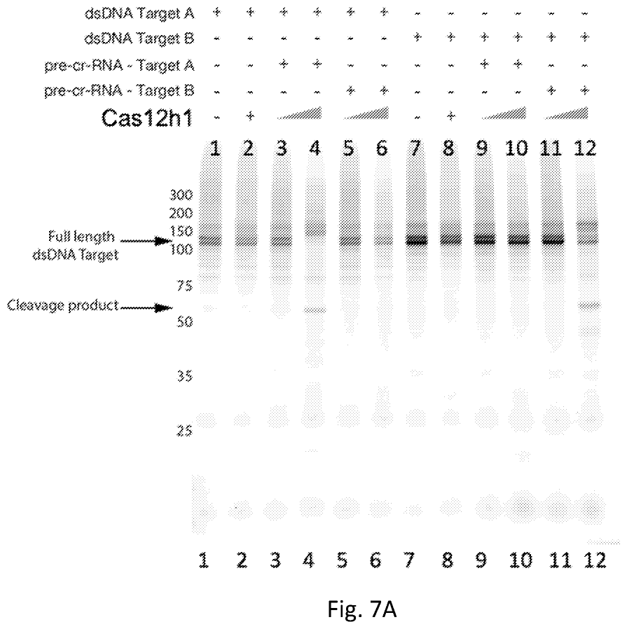

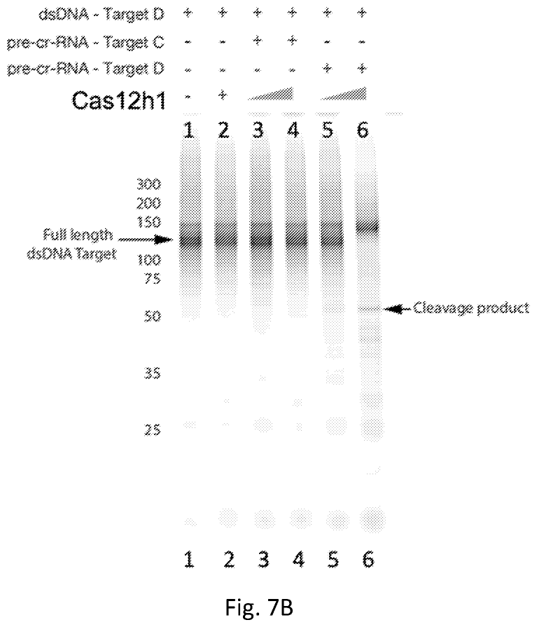

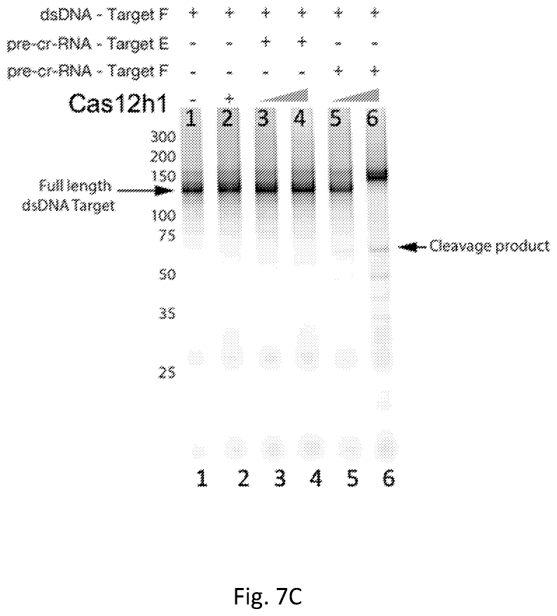

As shown in FIG. 7A, FIG. 7B, and FIG. 7C, target-specific cleavage was observed in each of the targets with its corresponding Cas12h1 RNP (e.g., lanes 4 and 12 of FIG. 7A, lane 6 of FIG. 7B, and lane 6 of FIG. 7C). Cleavage was positively correlated with Cas12h1 concentration, as shown in FIG. 7A, FIG. 7B, and FIG. 7C (e.g., the cleavage band was more pronounced in lane 4 of FIG. 7A than in lane 3). No detectable cleavage activity was observed in the absence of pre-crRNA (RNA guide) (e.g., lanes 2 and 8 of FIG. 7A, lane 2 of FIG. 7B, and lane 2 of FIG. 7C) and/or in the absence of Cas12h1 (e.g., lanes 1 and 7 of FIG. 7A, lane 1 of FIG. 7B, and lane 1 of FIG. 7C). Furthermore, no detectable cleavage activity was observed for Cas12h1 complexed with a non-targeting pre-crRNA (RNA guide). For example, no detectable cleavage was observed in Target A when using the pre-crRNA designed for Target B, and no detectable cleavage was observed in Target B when using the pre-crRNA designed for Target A (e.g., lanes 6 and 10 of FIG. 7A). Likewise, this pattern was consistent for Target D in the presence of non-targeting pre-crRNA designed for Target C (e.g., lane 4 of FIG. 7B) and for Target F in the presence of non-targeting pre-crRNA designed for Target E (e.g., lane 4 of FIG. 7C).

This suggests target-specific dsDNA cleavage activity by Cas12h1.

### Example 8—ssDNA Cleavage with Cas12h1

In this Example, Cas12h1 was evaluated for ssDNA cleavage activity.

ssDNA target cleavage assays were set up in reaction buffer (50 mM NaCl, 10 mM Tris, 10 mM MgCl₂, 1 mM DTT, pH 8.0) similar to the dsDNA assays described in Example 7. Negative controls with no Cas12h1 and non-target ssDNA were also tested.

Briefly, Cas12h1 protein was generated through an in vitro transcription-translation (IVTT) system. A dsDNA template for Cas12h1 including the promoter was amplified from the plasmid using PCR. To generate Cas12h1 protein, dsDNA template was incubated with an IVTT reagent. To generate an RNP complex of Cas12h1+pre-crRNA, dsDNA template was incubated with an IVTT reagent in the presence of 200 nM pre-crRNA (SEQ ID NO: 18).

The RNP complex was incubated with 500 nM pre-crRNA (SEQ ID NO: 18) in the assay buffer before adding near-infrared fluorescent dye labelled ssDNA of Target G (SEQ ID NO: 17) from Example 7 (and shown in FIG. 9B) and incubating. Negative control non-target ssDNA was incubated with a Cas12h1 RNP in a similar fashion. Reactions were first treated with RNase cocktail with incubation. Next, the reactions were treated with Proteinase K.

To detect ssDNA cleavage products, the reactions were analyzed on a 15% TBE-Urea gel and imaged on a fluorescent digital imaging system (LI-COR Biosciences).

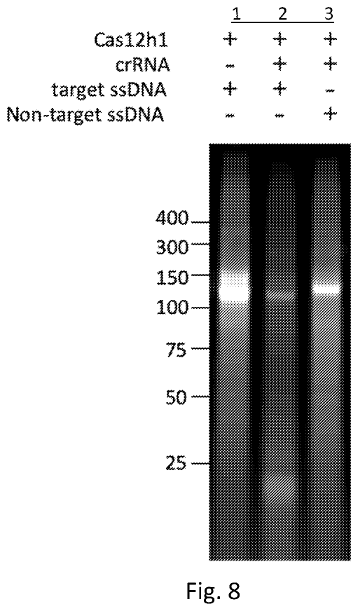

FIG. 8 shows an image of the TBE-Urea denaturing gel with the following reaction products: Lane 1: Target G ssDNA and Cas12h1 with no pre-crRNA, Lane 2: Target G ssDNA and Cas12h1 complexed with a top-strand (active orientation) pre-crRNA, and Lane 3: non-target ssDNA and Cas12h1 in complex with a top-strand (active orientation) pre-crRNA. As shown in the lane 2, Target G ssDNA showed detectable cleavage by Cas12h1 in the presence of its corresponding pre-crRNA in an active orientation. No detectable cleavage product was observed in the lanes 1 and 3, wherein pre-crRNA was not included or non-target ssDNA was used, respectively.

This suggests target-specific ssDNA cleavage activity by Cas12h1.

### Example 9—Targeting of Mammalian Gene by Cas12h1

This Example describes an indel assessment on a mammalian target by Cas12h1 introduced into mammalian cells by transient transfection.

Cas12h1 is cloned into a pcda3.1 backbone (Invitrogen). The plasmid is then maxi-prepped and diluted to 1 μg/μL. A mammalian target sequence adjacent to a 5′-RTR-3′, 5′-RTG-3′, 5′-NTG-3,′ or 5′-DHD-3′ PAM sequence is selected, and a corresponding RNA guide is designed as described herein. For RNA guide preparation, a dsDNA fragment encoding an RNA guide is derived by ultramers containing the target sequence scaffold, and the U6 promoter. Ultramers are resuspended in 10 mM Tris·HCl at a pH of 7.5 to a final stock concentration of 100 μM. Working stocks are subsequently diluted to 10 μM, again using 10 mM Tris·HCl to serve as the template for the PCR reaction. The amplification of the RNA guide is done in 50 μL reactions with the following components: 0.02 μL of aforementioned template, 2.5 μL forward primer, 2.5 μL reverse primer, 25 μL NEB HiFi Polymerase, and 20 μL water. Cycling conditions are: 1×(30 s at 98° C.), 30×(10 s at 98° C., 15 s at 67° C.), 1×(2 min at 72° C.). PCR products are cleaned up with a 1.8×SPRI treatment and normalized to 25 ng/μL.

Approximately 16 hours prior to transfection, 100 μL of 25,000 HEK293T cells in DMEM/10% FBS+Pen/Strep are plated into each well of a 96-well plate. On the day of transfection, the cells are 70-90% confluent. For each well to be transfected, a mixture of 0.5 μL of Lipofectamine 2000 and 9.5 μL of Opti-MEM is prepared and then incubated at room temperature for 5-20 minutes (Solution 1). After incubation, the lipofectamine:OptiMEM mixture is added to a separate mixture containing 182 ng of effector plasmid and 14 ng of crRNA and water up to 10 μL (Solution 2). In the case of negative controls, the crRNA is not included in Solution 2. The solution 1 and solution 2 mixtures are mixed by pipetting up and down and then incubated at room temperature for 25 minutes. Following incubation, 20 μL of the Solution 1 and Solution 2 mixture are added dropwise to each well of a 96 well plate containing the cells. 72 hours post transfection, cells are trypsinized by adding 10 μL of TrypLE to the center of each well and incubated for approximately 5 minutes. 100 μL of D10 media is then added to each well and mixed to resuspend cells. The cells are then spun down at 500 g for 10 minutes, and the supernatant is discarded. QuickExtract buffer is added to ⅕ the amount of the original cell suspension volume. Cells are incubated at 65° C. for 15 minutes, 68° C. for 15 minutes, and 98° C. for 10 minutes.

Samples for Next Generation Sequencing are prepared by two rounds of PCR. The first round (PCR1) is used to amplify specific genomic regions depending on the target. PCR1 products are purified by column purification. Round 2 PCR (PCR2) is done to add Illumina adapters and indexes. Reactions are then pooled and purified by column purification. Sequencing runs are done with a 150 cycle NextSeq v2.5 mid or high output kit.

Mean percent indels induced by Cas12h1 are measured in two bioreplicates and compared to values from negative control samples. A higher percentage of indels induced by Cas12h1, as compared to percent indels of negative control samples, is indicative of nuclease activity.

This Example shows how to evaluate Cas12h1 activity in mammalian cells.

SEQUENCES

SEQ ID NO: 1
[aquatic-non marine saline and alkaline-hypersaline lake sediment]
MKVHEIPRSQLLKIKQYEGSFVEWYRDLQEDRKKFASLLFRWAAFGYAAREDDGATYISPSQALLERRLL
LGDAEDVAIKFLDVLFKGGAPSSSCYSLFYEDFALRDKAKYSGAKREFIEGLATMPLDKIIERIRQDEQL
SKIPAEEWLILGAEYSPEEIWEQVAPRIVNVDRSLGKQLRERLGIKCRRPHDAGYCKILMEVVARQLRSH
NETYHEYLNQTHEMKTKVANNLTNEFDLVCEFAEVLEEKNYGLGWYVLWQGVKQALKEQKKPTKIQIAVD
QLRQPKFAGLLTAKWRALKGAYDTWKLKKRLEKRKAFPYMPNWDNDYQIPVGLTGLGVFTLEVKRTEVVV
DLKEHGKLFCSHSHYFGDLTAEKHPSRYHLKFRHKLKLRKRDSRVEPTIGPWIEAALREITIQKKPNGVF
YLGLPYALSHGIDNFQIAKRFFSAAKPDKEVINGLPSEMVVGAADLNLSNIVAPVKARIGKGLEGPLHAL
DYGYGELIDGPKILTPDGPRCGELISLKRDIVEIKSAIKEFKACQREGLTMSEETTTWLSEVESPSDSPR
CMIQSRIADTSRRLNSFKYQMNKEGYQDLAEALRLLDAMDSYNSLLESYQRMHLSPGEQSPKEAKFDTKR
ASFRDLLRRRVAHTIVEYFDDCDIVFFEDLDGPSDSDSRNNALVKLLSPRTLLLYIRQALEKRGIGMVEV
AKDGTSQNNPISGHVGWRNKQNKSEIYFYEDKELLVMDADEVGAMNILCRGLNHSVCPYSFVTKAPEKKN
DEKKEGDYGKRVKRFLKDRYGSSNVRFLVASMGFVTVTTKRPKDALVGKRLYYHGGELVTHDLHNRMKDE
IKYLVEKEVLARRVSLSDSTIKSYKSFAHV
SEQ ID NO: 2
MKVHEIPRSQLLKIKQYEGSFVEWYRDLQEDRKKFASLLFRWAAFGYAAREDDGATYISPSQALLERRLL
LGDAEDVAIKFLDVLFKGGAPSSSCYSLFYEDFALRDKAKYSGAKREFIEGLATMPLDKIIERIRQDEQL
SKIPAEEWLILGAEYSPEEIWEQVAPRIVNVDRSLGKQLRERLGIKCRRPHDAGYCKILMEVVARQLRSH
NETYHEYLNQTHEMKTKVANNLTNEFDLVCEFAEVLEEKNYGLGWYVLWQGVKQALKEQKKPTKIQIAVD
QLRQPKFAGLLTAKWRALKGAYDTWKLKKRLEKRKAFPYMPNWDNDYQIPVGLTGLGVFTLEVKRTEVVV
DLKEHGKLFCSHSHYFGDLTAEKHPSRYHLKFRHKLKLRKRDSRVEPTIGPWIEAALREITIQKKPNGVF
YLGLPYALSHGIDNFQIAKRFFSAAKPDKEVINGLPSEMVVGAAALNLSNIVAPVKARIGKGLEGPLHAL
DYGYGELIDGPKILTPDGPRCGELISLKRDIVEIKSAIKEFKACQREGLTMSEETTTWLSEVESPSDSPR
CMIQSRIADTSRRLNSFKYQMNKEGYQDLAEALRLLDAMDSYNSLLESYQRMHLSPGEQSPKEAKFDTKR
ASFRDLLRRRVAHTIVEYFDDCDIVFFEDLDGPSDSDSRNNALVKLLSPRTLLLYIRQALEKRGIGMVEV
AKDGTSQNNPISGHVGWRNKQNKSEIYFYEDKELLVMDADEVGAMNILCRGLNHSVCPYSFVTKAPEKKN
DEKKEGDYGKRVKRFLKDRYGSSNVRFLVASMGFVTVTTKRPKDALVGKRLYYHGGELVTHDLHNRMKDE
IKYLVEKEVLARRVSLSDSTIKSYKSFAHV
SEQ ID NO: 3
gtgctggccgctctcgctagagggaggtcagagcac
SEQ ID NO: 4
gtgctctgacctccctctagcgagagcggccagcac
SEQ ID NO: 5
aaacttaggacgacaaagtgtcgccttccagttcggtgatatacgggatctctttctcaaacagttttgc
accttccgtcaatgccgtcatggatccgtggtgatggtgatggtgaccttggtcaaatcggtgtttgttt
SEQ ID NO: 6
gtgctggccgctctcgctagagggaggtcagagcacacggcattgacggaaggtgcaaaactgtttgaga
aagtgctggccgctctcgctagagggaggtcagagcac
SEQ ID NO: 7
acggcattgacggaaggtgcaaaactgtttgagaaa
SEQ ID NO: 8
aaacttaggacgacaaagtgcagatgtatttcgctttaatggtacccgtggtcgcgtcaccggtaccctc
gcctttaatgataaatttcataccttcgacgtcgccttccagttcggtgaggtcaaatcggtgtttgttt
SEQ ID NO: 9
gtgctggccgctctcgctagagggaggtcagagcacaaatttatcattaaaggcgagggtaccggtgacg
cggtgctggccgctctcgctagagggaggtcagagcac
SEQ ID NO: 10
aaatttatcattaaaggcgagggtaccggtgacgcg
SEQ ID NO: 11
aaacttaggacgacaaagtgaaactgtttgagaaagagatcccgtatatcaccgaactggaaggcgacgt
cgaaggtatgaaatttatcattaaaggcgagggtaccggtgacgcgaccaggtcaaatcggtgtttgttt
SEQ ID NO: 12
gtgctggccgctctcgctagagggaggtcagagcacataaatttcataccttcgacgtcgccttccagtt
cggtgctggccgctctcgctagagggaggtcagagcac
SEQ ID NO: 13
ataaatttcataccttcgacgtcgccttccagttcg
SEQ ID NO: 14
aaacttaggacgacaaagtgaagtacccgagccacatcaaggatttctttaagagcgccatgccggaagg
ttatacccaagagcgtaccatcagcttcgaaggcgacggcgtgtacaagaggtcaaatcggtgtttgttt
SEQ ID NO: 15
gtgctggccgctctcgctagagggaggtcagagcacgtacgctcttgggtataaccttccggcatggcgc
tcgtgctggccgctctcgctagagggaggtcagagcac
SEQ ID NO: 16
gtacgctcttgggtataaccttccggcatggcgctc
SEQ ID NO: 17
tccatgtctcgttatacgctgtggttcgccaacgcactcagcaactactnnnnnnnnccgaacctgttca
ataagtgtcctgtttctataccannnnnnnnactactctcagcattgacagctagctcagtcctaggta
SEQ ID NO: 18
gtgctggccgctctcgctagagggaggtcagagcactggtatagaaacaggacacttattgaacaggttc
gggtgctggccgctctcgctagagggaggtcagagcac
SEQ ID NO: 19
tggtatagaaacaggacacttattgaacaggttcgg

What is claimed is:

1. A composition comprising:
(a) a nuclease or a nucleic acid encoding the nuclease, wherein the nuclease comprises an amino acid sequence with at least 80% identity to SEQ ID NO: 1; and
(b) an RNA guide or a nucleic acid encoding the RNA guide, wherein the RNA guide comprises a direct repeat sequence and a spacer sequence,
wherein the nuclease binds to the RNA guide, and wherein the spacer sequence binds to a target nucleic acid.

2. The composition of any previous claim, wherein the nuclease comprises an amino acid sequence set forth in SEQ ID NO: 1.

3. The composition of any previous claim, wherein the nuclease comprises a RuvC domain or a split RuvC domain.

4. The composition of any previous claim, wherein the nuclease comprises a catalytic residue (e.g., aspartic acid or glutamic acid).

5. The composition of any previous claim, wherein the composition does not include a tracrRNA.

6. The composition of any previous claim, wherein the direct repeat sequence comprises a nucleotide sequence with at least 95% sequence identity to SEQ ID NO: 3 or SEQ ID NO: 4.

7. The composition of any previous claim, wherein the direct repeat sequence comprises the nucleotide sequence set forth in SEQ ID NO: 3 or SEQ ID NO: 4.

8. The composition of any previous claim, wherein the spacer sequence comprises between 15 and 24 nucleotides in length.

9. The composition of any previous claim, wherein the target nucleic acid comprises a sequence complementary to a nucleotide sequence in the spacer sequence.

10. The composition of any previous claim, wherein the nuclease recognizes a protospacer adjacent motif (PAM) sequence, wherein the PAM sequence comprises a nucleotide sequence set forth as 5′-RTR-3′, 5′-RTG-3′, 5′-NTG-3,′ or 5′-DHD-3′, wherein “R” is A or G, “D” is A or G or T, and “N” is any nucleobase.

11. The composition of any previous claim, wherein the PAM sequence comprises a nucleotide sequence set forth as 5′-ATG-3′, 5′-GTG-3′, 5′-ATA-3′, or 5′-GTA-3′.

12. The composition of any previous claim, wherein the nuclease cleaves the target nucleic acid.

13. The composition of any previous claim, wherein the target nucleic acid is single-stranded DNA or double-stranded DNA.

14. The composition of any previous claim, wherein the composition comprises at least 10% greater enzymatic activity than a reference composition, e.g., at least 10% greater nuclease activity than a nuclease activity of a reference composition.

15. The composition of any previous claim, wherein the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

16. The composition of any previous claim, wherein the nucleic acid encoding the nuclease is codon-optimized for expression in a cell.

17. The composition of any previous claim, wherein the nucleic acid encoding the nuclease is operably linked to a promoter.

18. The composition of any previous claim, wherein the nucleic acid encoding the nuclease is in a vector.

19. The composition of any previous claim, wherein the vector comprises a retroviral vector, a lentiviral vector, a phage vector, an adenoviral vector, an adeno-associated vector, or a herpes simplex vector.

20. The composition of any previous claim, wherein the composition is present in a delivery composition comprising a nanoparticle, a liposome, an exosome, a microvesicle, or a gene-gun.

21. A cell comprising the composition of any previous claim.

22. The cell of claim 21, wherein the cell is a eukaryotic cell, e.g., a mammalian cell, e.g., a human cell.

23. The cell of any previous claim, wherein the cell is a prokaryotic cell.

24. A method of binding the composition of any previous claim to the target nucleic acid in a cell comprising:
(a) providing the composition; and
(b) delivering the composition to the cell,
wherein the cell comprises the target nucleic acid, wherein the nuclease binds to the RNA guide, and wherein the spacer sequence binds to the target nucleic acid.

25. A method of introducing an insertion or deletion into a target nucleic acid in a cell comprising:
(a) providing the composition of any previous claim; and
(b) delivering the composition to the cell,
wherein recognition of the target nucleic acid by the composition results in a modification of the target nucleic acid.

26. The method of claim 24 or claim 25, wherein delivering the composition to the cell is by transfection.

27. The method of any previous claim, wherein the cell is a eukaryotic cell.

28. The method of any previous claim, wherein the cell is a prokaryotic cell.

29. The method of any previous claim, wherein the cell is a human cell.


SEQUENCE LISTING (20 sequences)

SEQ ID NO: 1 - protein, 870 aa, Artificial Sequence
  Description of Artificial Sequence Synthetic polypeptide
MKVHEIPRSQLLKIKQYEGSFVEWYRDLQEDRKKFASLLFRWAAFGYAAREDDGATYISPSQALLERRLLLGDAEDVAIKFLDVLFKGGAPSSSCYSLFYEDFALRDKAKYSGAKREFIEGLATMPLDKIIERIRQDEQLSKIPAEEWLILGAEYSPEEIWEQVAPRIVNVDRSLGKQLRERLGIKCRRPHDAGYCKILMEVVARQLRSHNETYHEYLNQTHEMKTKVANNLTNEFDLVCEFAEVLEEKNYGLGWYVLWQGVKQALKEQKKPTKIQIAVDQLRQPKFAGLLTAKWRALKGAYDTWKLKKRLEKRKAFPYMPNWDNDYQIPVGLTGLGVFTLEVKRTEVVVDLKEHGKLFCSHSHYFGDLTAEKHPSRYHLKFRHKLKLRKRDSRVEPTIGPWIEAALREITIQKKPNGVFYLGLPYALSHGIDNFQIAKRFFSAAKPDKEVINGLPSEMVVGAADLNLSNIVAPVKARIGKGLEGPLHALDYGYGELIDGPKILTPDGPRCGELISLKRDIVEIKSAIKEFKACQREGLTMSEETTTWLSEVESPSDSPRCMIQSRIADTSRRLNSFKYQMNKEGYQDLAEALRLLDAMDSYNSLLESYQRMHLSPGEQSPKEAKFDTKRASFRDLLRRRVAHTIVEYFDDCDIVFFEDLDGPSDSDSRNNALVKLLSPRTLLLYIRQALEKRGIGMVEVAKDGTSQNNPISGHVGWRNKQNKSEIYFYEDKELLVMDADEVGAMNILCRGLNHSVCPYSFVTKAPEKKNDEKKEGDYGKRVKRFLKDRYGSSNVRFLVASMGFVTVTTKRPKDALVGKRLYYHGGELVTHDLHNRMKDEIKYLVEKEVLARRVSLSDSTIKSYKSFAHV

SEQ ID NO: 2 - protein, 870 aa, Artificial Sequence
  Description of Artificial Sequence Synthetic polypeptide
MKVHEIPRSQLLKIKQYEGSFVEWYRDLQEDRKKFASLLFRWAAFGYAAREDDGATYISPSQALLERRLLLGDAEDVAIKFLDVLFKGGAPSSSCYSLFYEDFALRDKAKYSGAKREFIEGLATMPLDKIIERIRQDEQLSKIPAEEWLILGAEYSPEEIWEQVAPRIVNVDRSLGKQLRERLGIKCRRPHDAGYCKILMEVVARQLRSHNETYHEYLNQTHEMKTKVANNLTNEFDLVCEFAEVLEEKNYGLGWYVLWQGVKQALKEQKKPTKIQIAVDQLRQPKFAGLLTAKWRALKGAYDTWKLKKRLEKRKAFPYMPNWDNDYQIPVGLTGLGVFTLEVKRTEVVVDLKEHGKLFCSHSHYFGDLTAEKHPSRYHLKFRHKLKLRKRDSRVEPTIGPWIEAALREITIQKKPNGVFYLGLPYALSHGIDNFQIAKRFFSAAKPDKEVINGLPSEMVVGAAALNLSNIVAPVKARIGKGLEGPLHALDYGYGELIDGPKILTPDGPRCGELISLKRDIVEIKSAIKEFKACQREGLTMSEETTTWLSEVESPSDSPRCMIQSRIADTSRRLNSFKYQMNKEGYQDLAEALRLLDAMDSYNSLLESYQRMHLSPGEQSPKEAKFDTKRASFRDLLRRRVAHTIVEYFDDCDIVFFEDLDGPSDSDSRNNALVKLLSPRTLLLYIRQALEKRGIGMVEVAKDGTSQNNPISGHVGWRNKQNKSEIYFYEDKELLVMDADEVGAMNILCRGLNHSVCPYSFVTKAPEKKNDEKKEGDYGKRVKRFLKDRYGSSNVRFLVASMGFVTVTTKRPKDALVGKRLYYHGGELVTHDLHNRMKDEIKYLVEKEVLARRVSLSDSTIKSYKSFAHV

SEQ ID NO: 3 - DNA, 36 bp, Artificial Sequence
  Description of Artificial Sequence Synthetic oligonucleotide
gtgctggccgctctcgctagagggaggtcagagcac

SEQ ID NO: 4 - DNA, 36 bp, Artificial Sequence
  Description of Artificial Sequence Synthetic oligonucleotide
gtgctctgacctccctctagcgagagcggccagcac

SEQ ID NO: 5 - DNA, 140 bp, Unknown
  Description of Unknown Target sequence
aaacttaggacgacaaagtgtcgccttccagttcggtgatatacgggatctctttctcaaacagttttgcaccttccgtcaatgccgtcatggatccgtggtgatggtgatggtgaccttggtcaaatcggtgtttgttt

SEQ ID NO: 6 - DNA, 108 bp, Artificial Sequence
  Description of Artificial Sequence Synthetic polynucleotide
gtgctggccgctctcgctagagggaggtcagagcacacggcattgacggaaggtgcaaaactgtttgagaaagtgctggccgctctcgctagagggaggtcagagcac

SEQ ID NO: 7 - DNA, 36 bp, Artificial Sequence
  Description of Artificial Sequence Synthetic oligonucleotide
acggcattgacggaaggtgcaaaactgtttgagaaa

SEQ ID NO: 8 - DNA, 140 bp, Unknown
  Description of Unknown Target sequence
aaacttaggacgacaaagtgcagatgtatttcgctttaatggtacccgtggtcgcgtcaccggtaccctcgcctttaatgataaatttcataccttcgacgtcgccttccagttcggtgaggtcaaatcggtgtttgttt

SEQ ID NO: 9 - DNA, 108 bp, Artificial Sequence
  Description of Artificial Sequence Synthetic polynucleotide
gtgctggccgctctcgctagagggaggtcagagcacaaatttatcattaaaggcgagggtaccggtgacgcggtgctggccgctctcgctagagggaggtcagagcac

SEQ ID NO: 10 - DNA, 36 bp, Artificial Sequence
  Description of Artificial Sequence Synthetic oligonucleotide
aaatttatcattaaaggcgagggtaccggtgacgcg

SEQ ID NO: 11 - DNA, 140 bp, Unknown
  Description of Unknown Target sequence
aaacttaggacgacaaagtgaaactgtttgagaaagagatcccgtatatcaccgaactggaaggcgacgtcgaaggtatgaaatttatcattaaaggcgagggtaccggtgacgcgaccaggtcaaatcggtgtttgttt

SEQ ID NO: 12 - DNA, 108 bp, Artificial Sequence
  Description of Artificial Sequence Synthetic polynucleotide
gtgctggccgctctcgctagagggaggtcagagcacataaatttcataccttcgacgtcgccttccagttcggtgctggccgctctcgctagagggaggtcagagcac

SEQ ID NO: 13 - DNA, 36 bp, Artificial Sequence
  Description of Artificial Sequence Synthetic oligonucleotide
ataaatttcataccttcgacgtcgccttccagttcg

SEQ ID NO: 14 - DNA, 140 bp, Unknown
  Description of Unknown Target sequence
aaacttaggacgacaaagtgaagtacccgagccacatcaaggatttctttaagagcgccatgccggaaggttatacccaagagcgtaccatcagcttcgaaggcgacggcgtgtacaagaggtcaaatcggtgtttgttt

SEQ ID NO: 15 - DNA, 108 bp, Artificial Sequence
  Description of Artificial Sequence Synthetic polynucleotide
gtgctggccgctctcgctagagggaggtcagagcacgtacgctcttgggtataaccttccggcatggcgctcgtgctggccgctctcgctagagggaggtcagagcac

SEQ ID NO: 16 - DNA, 36 bp, Artificial Sequence
  Description of Artificial Sequence Synthetic oligonucleotide
gtacgctcttgggtataaccttccggcatggcgctc

SEQ ID NO: 17 - DNA, 139 bp, Unknown
  Description of Unknown Target sequence
  modified_base (50)..(57): a, c, t, g, unknown or other
  modified_base (94)..(101): a, c, t, g, unknown or other
tccatgtctcgttatacgctgtggttcgccaacgcactcagcaactactnnnnnnnnccgaacctgttcaataagtgtcctgtttctataccannnnnnnnactactctcagcattgacagctagctcagtcctaggta

SEQ ID NO: 18 - DNA, 108 bp, Artificial Sequence
  Description of Artificial Sequence Synthetic polynucleotide
gtgctggccgctctcgctagagggaggtcagagcactggtatagaaacaggacacttattgaacaggttcgggtgctggccgctctcgctagagggaggtcagagcac

SEQ ID NO: 19 - DNA, 36 bp, Artificial Sequence
  Description of Artificial Sequence Synthetic oligonucleotide
tggtatagaaacaggacacttattgaacaggttcgg

SEQ ID NO: 20 - RNA, 36 nt, Artificial Sequence
  Description of Artificial Sequence Synthetic oligonucleotide
gugcuggccgcucucgcuagagggaggucagagcac